(19) United States
(12) Patent Application Publication
(10) Pub. No.: US 2022/0051547 A1
(43) Pub. Date: Feb. 17, 2022

(54) Sanitization Analysis Devices, Systems, and Methods

(71) Applicant: Hygenius, Inc., Davis, CA (US)

(72) Inventor: Sam Michael Skinner, Davis, CA (US)

(73) Assignee: Hygenius, Inc., Davis, CA (US)

(21) Appl. No.: 17/402,464

(22) Filed: Aug. 13, 2021

Related U.S. Application Data
(60) Provisional application No. 63/065,764, filed on Aug. 14, 2020.

(51) Int. Cl. G08B 21/24; G06K 9/00; G09B 19/00; A61L 2/00; A61L 2/24; A61L 2/28

(52) U.S. Cl. CPC G08B 21/245; G06K 9/00536; G06K 9/00355; G09B 19/0076; G06T 7/254; A61L 2/24; A61L 2/28; G06T 2207/10064; G06T 2207/30196; A61L 2/0088

(57) ABSTRACT

Systems, devices, methods, and software of the present invention provide for sanitization monitoring of hands, other body parts, and objects. The systems and devices including a detector to provide images of the object within its detection range, and at least one processor to receive the images from the detector, determine areas of the image corresponding to sanitized areas of the object from unsanitized areas of the object, calculate a percentage of sanitized areas to the total area corresponding to the sanitized and unsanitized areas, and report at least the percentage of sanitized area. In various embodiments, users sanitize their hands with fluorescing hand sanitizer and/or a fluorescing germ-proxy agent with soap and water and the sanitized and unsanitized areas are determined based on the amount of fluorescing material remaining on the hands after application.

### CROSS-REFERENCE TO RELATED APPLICATIONS

This application claims priority to and the benefit of U.S. Provisional Patent Application No. 63/065,764 filed on Aug. 14, 2020, which is incorporated by reference in its entireties.

### STATEMENT REGARDING FEDERALLY SPONSORED RESEARCH OR DEVELOPMENT

Not Applicable

### BACKGROUND OF THE INVENTION

### Field of the Invention

The present invention generally relates to analyzing cleanliness/sanitization of body parts and other objects. More specifically, the invention relates to sanitization monitoring and analysis devices, systems, and methods for analyzing the sanitization of hands and other objects to support sanitized procedures in the life sciences, e.g., medicine, and other areas of life where sanitization is of importance.

### BACKGROUND ART

1 in 25 hospitalized patients in the US are affected by a healthcare-associated infection (HAI) acquired during their stay in a medical facility as a result of poor infection prevention efforts, which cause up to $31 billion in medical costs each year in treatment in the US alone according to “Healthcare-Associated Infections.” Healthy People, 2020, http://www.healthypeople.gov/2020/topics-objectives/topic/healthcare-associated-infections. The number one cause of HAI is poor hand hygiene, which directly accounts for at least 40%, and up to even 70% of all HAIs according to “Hand Hygiene Compliance and Associated Factors among Health Care Providers in Central Gondar Zone Public Primary Hospitals, Northwest Ethiopia.”, Engdaw, Garedew Tadege, et al., Antimicrobial Resistance & Infection Control, vol. 8, no. 1, 2019, doi:10.1186/s13756-019-0634-z., and Pyrek, Kelly M., Infection Control Today, Informa Exhibitions LLC, 2014, The Economics of Hand Hygiene Compliance Monitoring., respectively.

Hand sanitization with a sanitizer solution and hand washing with soap & water are the best ways to keep one's hands clean, and kill or reduce bacteria and viruses on hands. Hand sanitizer manufacturers claim that their products can kill 99.9% of germs, with studies showing that proper hand sanitizer application can eliminate nearly all pathogens on one's hands (Sutter, S. Tschudin, et al. “Effect of Teaching Recommended World Health Organization Technique on the Use of Alcohol-Based Hand Rub by Medical Students.” Infection Control & Hospital Epidemiology, vol. 31, no. 11, 2010, pp. 1194-1195., doi:10.1086/656745. https://edoc.unibas.ch/23522/1/effect_of_teaching_recommended_world_health_organization_technique_on_the_use_of_alcoholbased_hand_rub_by_medicalstudents.pdf), at least most of them (over 80%, per Chow, Angela, et al. “Alcohol Handrubbing and Chlorhexidine Handwashing Protocols for Routine Hospital Practice: A Randomized Clinical Trial of Protocol Efficacy and Time Effectiveness.” American Journal of Infection Control, vol. 40, no. 9, 2012, pp. 800-805., doi:10.1016/j.ajic.2011.10.005. https://www.sciencedirect.com/science/article/pii/S0196655311012594, and Girou, E. “Efficacy of Handrubbing with Alcohol Based Solution versus Standard Handwashing with Antiseptic Soap: Randomised Clinical Trial.” Bmj, vol. 325, no. 7360, 2002, pp. 362-362., doi:10.1136/bmj.325.7360.362. https://www.ncbi.nlm.nih.gov/pmc/articles/PMC117885/).

Hand washing also reduces pathogens on hands to a high degree, with 15 seconds of proper washing reducing about 90% of pathogens, and 30 seconds up to 99.9% (Harvard Health Publishing, The Handiwork of Good Health, www.health.harvard.edu/newsletter_article/The_handiwork_of_good_health.). However, oftentimes this high reduction factor of pathogens is not the case.

Hand sanitizer and soap & water are just tools people can use to reduce germ levels, but the effectiveness of those tools depends on how well the tools are used. Unfortunately, not very many people apply hand sanitizer and/or wash their hands properly and effectively.

There are two main barriers to effective use of hand sanitizer and soap & water. The first is called the “Hawthorne Effect”, something even Healthcare Workers (HCWs) fall prey to, whereby people change their behavior depending on whether or not they know they're being monitored. Investigators found HCWs only complied with hand hygiene protocols, i.e., only sanitized or washed their hands after medical center guidelines said they should, only 22% of the time when they didn't think they were being observed (Barzilay, Julie. “Doctors' Hand Hygiene Plummets Unless They Know They're Being Watched, Study Finds.” ABC News, ABC News Network, 9 Jun. 2016, https://abcnews.go.com/Health/doctors-hand-hygiene-plummets-watched-study-finds/story?id=39737505).

The second barrier is the lack of training on and reliable/consistent use of proper hand cleaning techniques. For both hand sanitization and hand washing, the World Health Organization (“WHO”) has laid out a 6-step protocol that cleans all parts of an individual's hands through tactical hand rub techniques. See Widmer, Andreas F., et al. “Introducing Alcohol-Based Hand Rub for Hand Hygiene The Critical Need for Training.” Infection Control & Hospital Epidemiology, vol. 28, no. 1, 2007, pp. 50-54., doi:10.1086/510788. https://www.researchgate.net/profile/Reno_Frei/publication/6573072_Introducing_Alcohol-Based_Hand_Rub_for_Hand_Hygiene_The_Critical_Need_for_Training/links/00463514c7b95a8561000000/Introducing- Alcohol-Based-Hand-Rub-for-Hand- Hygiene-The-Critical-Need-for-Training.pdf, which is incorporated herein by reference However, one study showed that again, even among highly trained and very health-conscious healthcare workers, only 31% used proper technique, which correlated to the hand cleaning efforts only being about 66% as effective as they would have been had proper technique been used. This is due to the fact that proper use of such techniques has been proven to increase the microbial reduction factor of hand cleaning by 50% compared to non-use.

Thus, in a very real way, proper use of hand sanitizer and soap & water is very important to public health. As such, there is a continuing need for sanitization methods, solutions, and devices that improve the proper use of hand sanitizer and soap & water and other hygiene methods.

### BRIEF SUMMARY OF THE INVENTION

The present invention addresses the above noted needs by providing sanitizing monitoring devices, systems and methods that may be employed in a stand-alone manner or with existing sanitizing devices, such as hand sanitizer stations, sinks, etc. to provide real time feedback to the person performing the sanitization of hands, other body parts, other devices, etc., and data for improvement. Systems, devices, methods, and software of the present invention provide for sanitization monitoring of hands, other body parts, and object. The systems and devices including a detector to provide images of the object within its detection range, and at least one processor to receive the images from the detector, determine areas of the image corresponding to sanitized areas of the object from unsanitized areas of the object, calculate a percentage of sanitized areas to the total area corresponding to the sanitized and unsanitized areas, and report at least the percentage of sanitized area. In various embodiments, the sanitizing monitoring device analyzes and quantifies the thoroughness of the sanitization into a score as the sanitization is performed and/or afterward, and provide feedback in the form of suggestions for improvement and areas where additional sanitization may be required.

In various embodiments, the system may include a chamber with a black interior with UV LED lights positioned the inside of the chamber to illuminate a hand detection area in the chamber. The detector may be positioned to detect hands placed in the hand detection area, which may be implemented as visible light camera suitable for capturing images and streaming video images. The system includes a computer for receiving and processing the image data from the detector and. receive the images from the detector, determining areas of the image corresponding to sanitized areas of the hands from unsanitized areas of the hands based on the fluorescence of the hands, calculating a score using at least a percentage of the sanitized areas to a total area corresponding to the sanitized plus the unsanitized areas, and providing at least the score to a display positioned proximate the chamber to display at least the score. Additional information about how to improve handwashing scores may also be provided to the user.

In various other embodiments, an illumination device may or may not be employed when the detector includes a thermal imaging camera that may be used alone or with a visible light camera to provide thermal images of the hands or other objects. In thermal imaging camera embodiments, sanitization may be performed in the hand detection area using alcohol-based hand sanitizer and the thermal imaging camera is used to detect surface temperature differences induced by the hand sanitizer to determine the sanitized and unsanitized areas of the hands or object.

In various embodiments, the system may include a hand sanitizer dispenser containing fluorescing hand sanitizer and/or a fluorescing germ-proxy agent and soap dispenser positioned proximate the chamber. The computer, via one or more processors, may provide instructions on using the system to sanitize their hands with the fluorescing hand sanitizer and a fluorescing germ-proxy agent with soap to the display for viewing and suggestions for improvement. The software running on the computer may also detect one or more hand rubbing techniques, as well as the duration of hand rubbing from the images, which may also be used to calculate the calculate the score.

The system may identify a user of the system based on information from an identification device, such as an RFID device, information that may be manually input into the display by the user, and an identifier received via a wireless signal. The processor would then store information derived from the sanitization process, such as the scores, the percentage of sanitized area, the detected hand rubbing techniques, the duration of hand rubbing, the number of times sanitization was attempted, etc. and provide some or all of the information to the user via the display and/or to interested parties and management systems for storage that are remote from the system.

The system via the software and processors may compare the sanitization process information against other users and/or various groups of users and provide the comparison of information to the display for viewing. The system may also request, via the display, the user to further sanitize their hands when the score does not exceed a threshold score. When the process is complete, the system may associate the information with the user and store the information associated with the user locally in the computer and/or remote in a database that may be associated with a management system.

In various embodiments, the system may include a water faucet to dispense water proximate the hand detection area to support the washing of hands and other objects in the hand detection area of the system.

The system and software of the present invention may implement various methods of hand sanitization monitoring by providing a chamber with at least one illumination device positioned within the chamber to illuminate a hand detection area within the chamber and a detector positioned within the chamber to detect and provide images of hands within the hand detection area. A user may sanitize their hands with at least one of a fluorescing hand sanitizer and a fluorescing germ-proxy agent with soap and perform the sanitization in the hand detection area of the chamber, while the detection area is illuminated by the at least one illumination device; such as a UV light and the detector, such as a visual camera, may be detecting and providing images of the hands to a processor in the computer. The processor may analyze the images and determine areas of the image corresponding to sanitized areas and unsanitized areas of the hands, then calculate a percentage score using the total sanitized area divided by the total area corresponding to the total sanitized plus total unsanitized areas, and provide the score and/or percentage of sanitized area to the display and/or the management system.

For embodiments in which the hand sanitization is performed in the hand detection area, the software and system may detect various hand rubbing techniques and the duration of hand rubbing from the images and use that information in the calculation of the score, as well as report the information to the user along with information about how the user can improve their sanitization score. In various embodiments, the scores of various users may be compared and displayed to incentivize improve sanitization habits.

Accordingly, the present disclosure addresses the continuing need for improved methods and system for sanitizing hands and other objects and methods for documenting and continually improve hygiene.

### BRIEF DESCRIPTION OF THE DRAWINGS

The accompanying drawings are included for the purpose of exemplary illustration of various aspects and embodiments of the present invention, and not for purposes of limiting the invention, wherein:

The present invention is illustrated by way of example and not limitation in the figures of the accompanying drawings in which like references indicate similar elements.

FIG. 1 illustrates an angled view of the front right side of exemplary embodiments of the system.

FIG. 2 illustrates an angled view of the front left side of exemplary embodiments of the system.

FIG. 3 illustrates a view that shows the front, left, and underside of exemplary embodiments of the system.

FIG. 4 illustrates a top view of exemplary embodiments of the system.

FIG. 5 illustrates a bottom view of exemplary embodiments of the system.

FIG. 6 illustrates a front view of exemplary embodiments of the system.

FIG. 7 illustrates a back view of exemplary embodiments of the system.

FIG. 8 illustrates a view of the left side of exemplary embodiments of the system.

FIG. 9 illustrates a view of the right side of exemplary embodiments of the system.

FIG. 10 illustrates a view of the top, back, and right sides of exemplary embodiments of the system.

FIG. 11 illustrates a view of the top, front, and right sides of exemplary embodiments of the system.

FIG. 12 illustrates a view of the top, back, and right sides of exemplary embodiments of the system.

FIG. 13 illustrates a front view of other exemplary embodiments of the system.

FIG. 14 illustrates a top, right, front view of other exemplary embodiments of the system.

FIG. 15 illustrates a bottom side view of other exemplary embodiments of the system.

FIG. 16 illustrates a top, left, back view of other exemplary embodiments of the system.

FIG. 17 illustrates a back view of other exemplary embodiments of the system.

FIG. 18 illustrates a back view of other exemplary embodiments of the system.

FIG. 19 shows exemplary ecosystems in which the system may be employed.

FIG. 20 depicts exemplary component process flows that may be employed in the system.

FIGS. 21 and 22 provide exemplary software process flows.

FIGS. 23-34 show exemplary displays and techniques involved in the sanitization monitoring and analysis process.

FIG. 35 illustrates exemplary component embodiments of various computing resources that may be used in the present invention.

In the drawings and detailed description, the same or similar reference numbers may identify the same or similar elements. It will be appreciated that the implementations, features, etc. described with respect to embodiments in specific figures may be implemented with respect to other embodiments in other figures, unless expressly stated, or otherwise not possible.

### DETAILED DESCRIPTION OF THE INVENTION

Systems 15 of the present invention provide for sanitization monitoring and may include cleaning of hands, other body parts, objects, etc. The system 15 may include various components that may be integrated as a stand-alone device 17 or separately interoperating to provide the desired functionality.

To ease the description of the invention, the present invention will be described in terms of sanitization, which should be interpreted in the context of the present invention and application to mean cleaning, sanitization, disinfection, sterilization, and other similar terms pertaining to processes and procedures for removing and/or killing microorganism and non-living matter present on a surface, unless otherwise stated. To further ease the description of the invention, the system 15 and its various features, embodiments, etc. will be described with respect to sanitizing and monitoring and determining the sanitization/cleanliness of hands. However, it will be appreciated that the invention may also be more generally applicable to sanitization monitoring of other body parts, objects, etc. with appropriate modification to the dimensions and components of the system, unless otherwise stated.

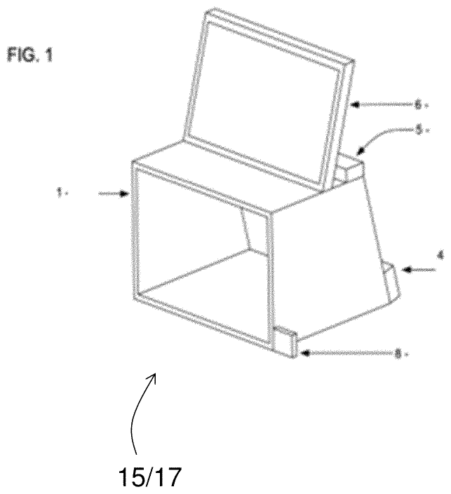

FIG. 1 depicts exemplary embodiments of system 15 as shown from a front, right, and top view. The system 15 may include a chamber 1 that may be any shape or design conducive to enable hands, other body parts, or workpieces and objects to be examined within the chamber 1. For example, the chamber 1 may be a rectangular prism in shape with a slanted back wall, with the entire front as an opening for a user to insert both hands and possibly perform sanitization activities within the chamber.

The interior of the chamber 1 may be a dark, non-reflective color, such as black, but may not be limited to such a specific color or lighting. The use of the chamber 1, while not required in many embodiments, provides a consistent environment for hand detection and the elimination of the background from the analysis.

A power source 4 may be provided in the form one or more batteries deployed on or in proximity to the chamber 1. Other embodiments of the power source 4 may include a power cable connected to the device and draws power from a wall outlet or other electricity source, in addition to, or in lieu, of the batteries.

The system 15 may include a computer 5, which may be integrated into or attached to the inside or outside wall of the chamber 1 or separate from the chamber 1. FIG. 1 depicts the computer 5 mounted to the top outside wall of the chamber 1. The computer 5 may be a single board computer that may include memory and storage that may be self-contained or interoperate with a remote server and/or management system.

A display 6 may be provided proximate to the chamber to provide feedback and information to the users and/or others. The display may be a monitor, television, etc. and may include analog display, red/yellow/green lights, etc. FIG. 1 shows the display on the outside top of the chamber 1, but the display may be provided separately from the chamber 1. For example, the computer 5 and display 6 may be embodied as a desktop, laptop, tablet, phone, etc. that is deployed on or proximate the chamber 1 if it is desired to have the display 6 visible to the user. In other embodiments, the display may not be visible to the user or disabled, such as during testing to determine the baseline and/or periodic levels of sanitization.

In various embodiments, a user identification device 8 may be employed in the system 15 to identify the person using the system 15. For example, a Near Field Communications/Radio Frequency Identification (NFC/RFID) reader may be used to transfer data between user badges/devices and the system 15. In other embodiments, a camera may be used for visual identification, or a scanner for barcode/QR identification. In FIG. 1 embodiments, the id reader 8 is shown attached to the bottom front right corner of the chamber 1, but may be deployed in any location suitable on or separate from the chamber 1 for its purpose.

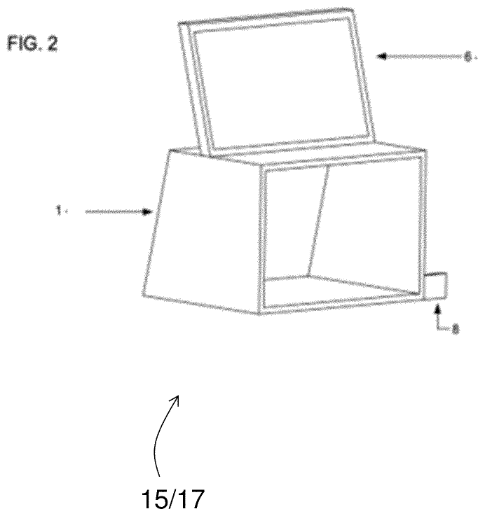

FIG. 2 shows another rotated view of the FIG. 1 embodiments showing the front, left, and top of the chamber 1 and exemplary location of the display 6 and id device 8.

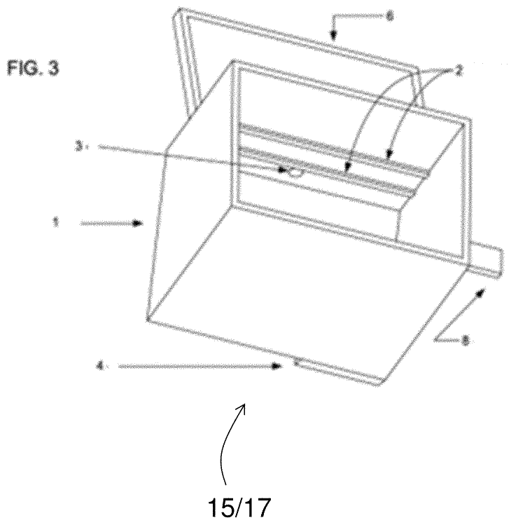

FIG. 3 illustrates various embodiments of the system 15, such as those in FIGS. 1-2, from a bottom left perspective of the chamber 1. This perspective depicts the outside bottom and left walls of the chamber 1, as well as its interior, and exemplary locations for the power source 4, display 6, and id device 8.

In various embodiments, one or more illumination devices, e.g., UV lights, 2 and a detector 3, such as a camera, may be provided inside the chamber 1, such as fitted to the inside top surface of the chamber 1 to visually detect a sanitization indicator. While UV LEDs are good for power efficiency and brightness, other embodiments of the system 15 may employ other types of UV lights or other types of illumination devices 2. In addition, more or less lights than shown in the figures may be employed. The illumination device may emit light in the UV A range, e.g., 395-405 nm, and at safe intensity levels.

The detector 3 may be a visual light camera, a thermal camera, or other appropriate detector suitably matched to detect an indicator being used in various embodiments. In FIG. 3 embodiments, the detector 3 may be located proximate the UV LED lights, such as near the back of the chamber's ceiling, though other embodiments may have it placed elsewhere.

In various embodiments employing a thermal sensor as the detector 3, the system 15 may not include a chamber 1 or lights 2. In these embodiments, the user may be instructed 1) to position their hands, object, etc. being sanitized proximate to the detector 3, so the detector 3 can detect a pre-clean thermal pattern, 2) apply hand sanitizer or other cleaner and clean their hands, objects, etc., and 3) place their hands, objects, etc. The system 15 then compares the pre-clean and post-clean images to determine the sanitization level. Some embodiments may not use a pre-clean image to determine sanitization level, which may be determined from the post-cleaning step alone.

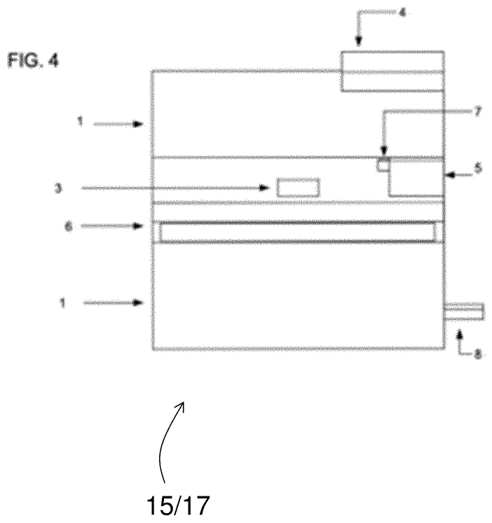

FIG. 4 illustrates a top view of embodiments corresponding to FIGS. 1-3, with the system 15 front facing down and back facing up. FIG. 4 further depicts a wireless receiver/transceiver 7, such as a Bluetooth transceiver in communication with the computer 5 to enable wireless communications between the computer 5 and one or more of the detector 3, id device 8, lights 3, display 6, and a remote management system. The wireless receiver 7 may also be an alternative to the id device 8.

FIG. 4 shows the detector 3 integrated into the back of the top wall of the chamber 1 behind the display 6 and to the left of the computer 5. However, as with component, the detector 3 may have it placed elsewhere on the chamber 1.

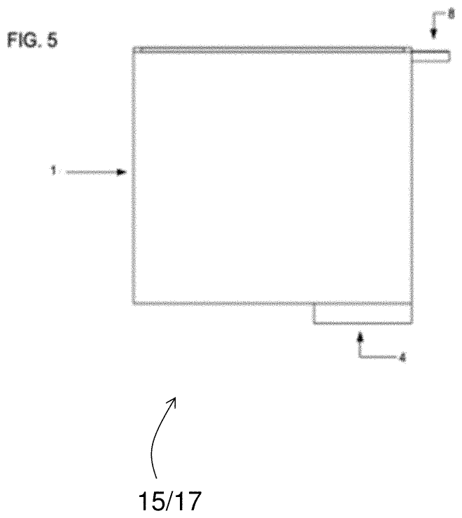

FIG. 5 illustrates a bottom view of the embodiments shown in FIGS. 1-4. In various other embodiments, it may be desirable to not include a bottom on the chamber 1. However, the bottom of the chamber 1 may provide a consistent background for the detector 3 in the hand detection area, which may enable the software to make consistently subtract/remove the bottom/background from images of the hands. In other embodiments not involving a chamber 1, it may also be desirable to provide a consistent background for the detector 3.

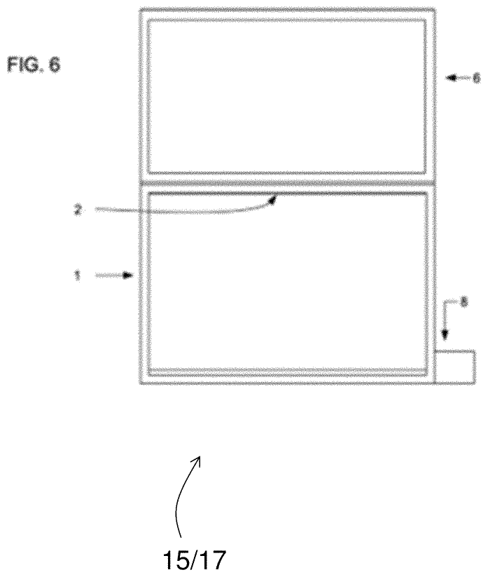

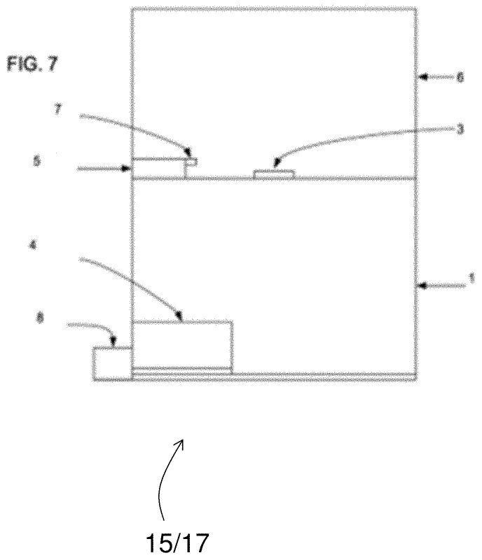

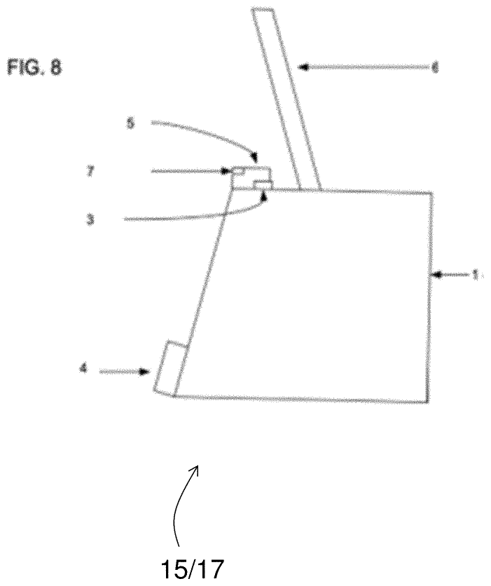

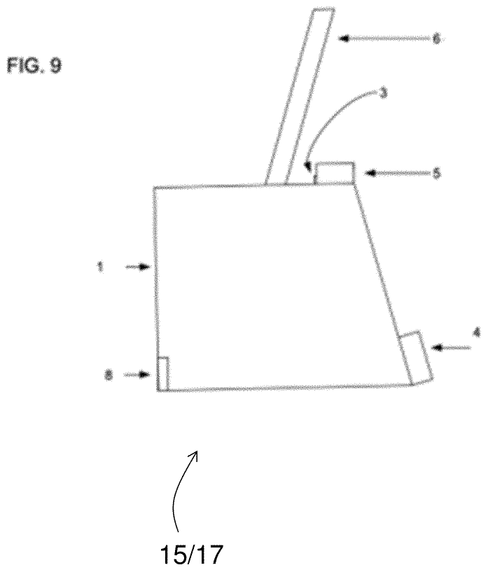

FIGS. 6-9 respectively show front, back, left, and right side view of the embodiments shown as perspective views in FIGS. 1-5. Of note, in FIGS. 8 and 9 are the angles/slants of the chamber 1 back wall and the display 6. The back wall of the chamber 1 is slanted back from top to bottom in this embodiment to be out of view of the detector, as well as provide users with extra room within the chamber 1 to clean their hands, etc., though other embodiments may have this slant be different or nonexistent.

The display 6 is slanted back from bottom to top in this embodiment in order to provide a better viewing angle for the user, though other embodiments may have this slant be different or nonexistent. Supporting pillars/walls as well as attachments at the base into the top wall of the chamber 1 are not shown in order to view the important components of the system 15 more easily.

FIG. 10 is a perspective view of the embodiments of FIGS. 1-9 from the top, back, right side. This illustration's view is important as it shows the 3 walls of the chamber (1) on which all of the external components (1,3-8) are placed, and their relative positions, as well as the slants of the back wall of the chamber and the display screen (6). The only components that cannot be seen are on the inside of the chamber integrated into its ceiling, namely the UV LED lights (2) and the underside/front of the optical device (3).

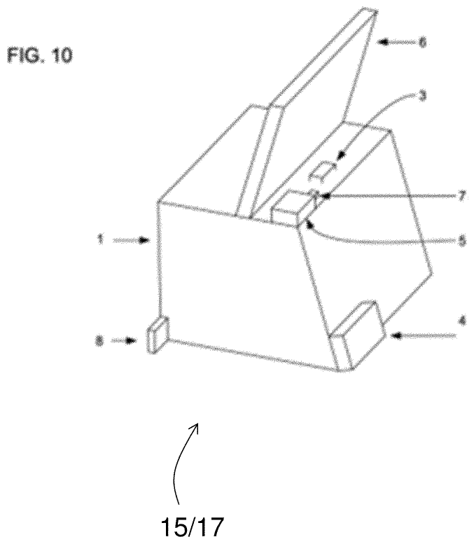

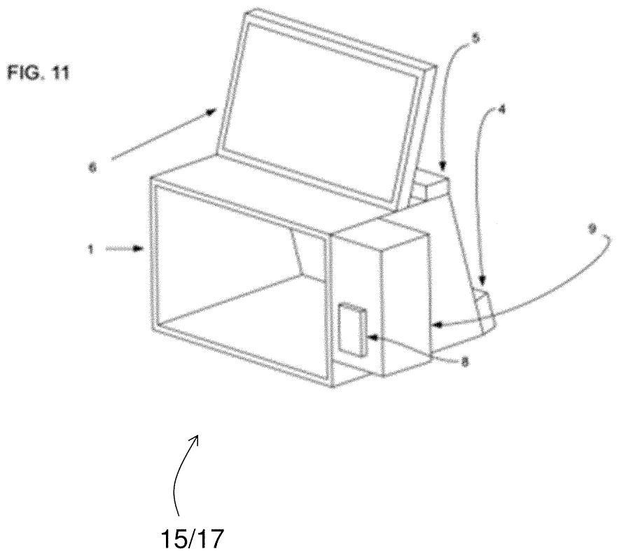

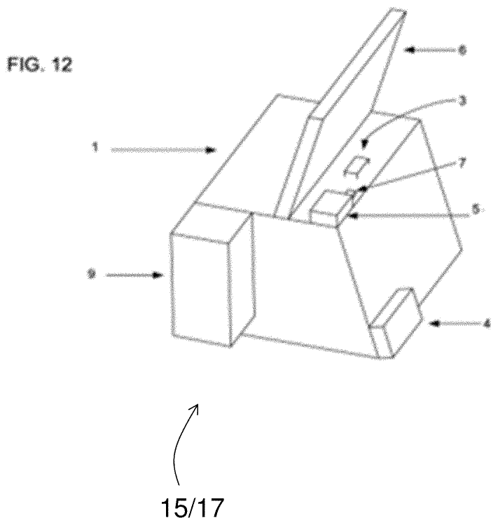

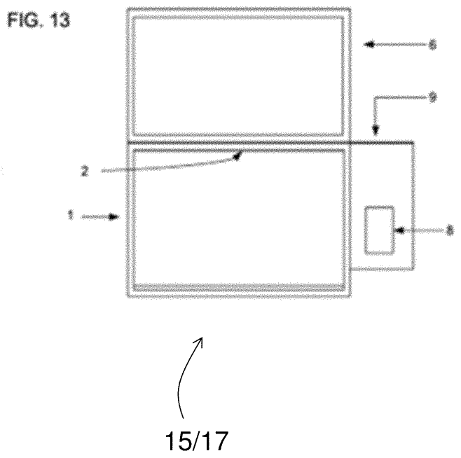

FIGS. 11-13 depict embodiments of the system 15 similar to FIGS. 1-10, but further includes a hand sanitizer dispenser 9 attached to the chamber 1. In these embodiments, the dispenser 9 is shown as attached to the right wall of the chamber 1, but may be positioned in other locations on the chamber 1. The dispenser 9 may be a manual or automatic, but it may be more desirable to employ an automatic dispenser 9 to minimize contact with the dispenser 9. The embodiments depicted in FIG. 11-13 also show the id device 8 positioned on the dispenser 9, but if may be located elsewhere on the chamber 1 or not on the chamber.

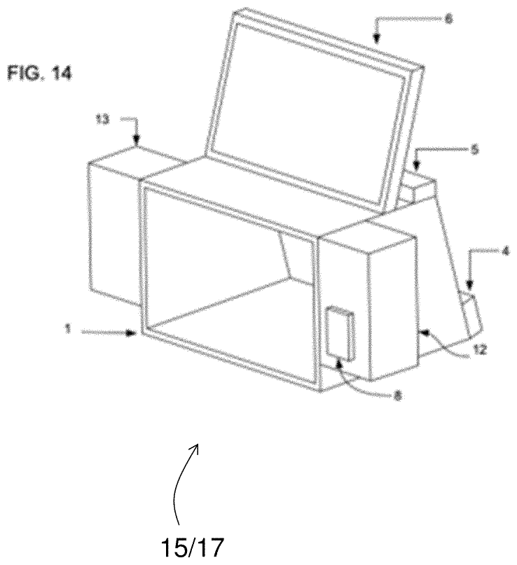

FIGS. 14-18 depict embodiments of the system 15 similar to the embodiments shown in FIGS. 1-10, but including an automatic sink faucet 11, an automatic fluorescing agent (germ-proxy) dispenser 12 connected to the right wall of the chamber 1, and an automatic soap dispenser 13. The id device 8 may be connected to the front of the automatic hand fluorescing agent dispenser 12, but may be positioned in other locations on the chamber 1 or separate from the chamber 1.

The dispensers 12 and 13 may positioned on opposite sides of the chamber 1 or be separate, such as may be in FIGS. 1-10. The dispensers may be automatic and shaped like rectangular prisms that dispense from the bottom, automatic dispensers shaped like cylinders that dispense from the top, or may be the kind that siphon the fluids from a tub hidden underneath a sink counter connected by a thin & long hose/tube, or something else entirely. In this embodiment the sink faucet and dispensers are as described, though other embodiments may have them shaped, placed, and oriented otherwise.

FIG. 15 is a bottom left front view showing a sink faucet integrated, showing a faucet spout 11 running along the ceiling of the wall of the chamber 1 and the faucet pipe end at the back bottom of the chamber 1, but the faucet 11 and plumbing may be placed elsewhere in the chamber 1. The detector 3 may be repositioned in these embodiments for a couple of reasons. One is to allow the detector 3 to be attached or embedded within the faucet 11 without impeding the flow of water to the opening of the faucet 11. The second and more important reason (hence why the detector 3 is not just placed underneath the faucet spout 11 but behind the opening) is that the sink is automatic, and should not always be running when one is using the hand rub techniques when washing their hands, and scanning their hands after completing their hand cleaning event. The detector 3 being in front of the faucet opening and motion sensor 10 allows one to have their hand cleaning and hand scanning monitored without activating the flow of water all the time. In this embodiment the sink faucet 11 and integrated components are as described, though other embodiments may have them shaped, placed, and oriented otherwise.

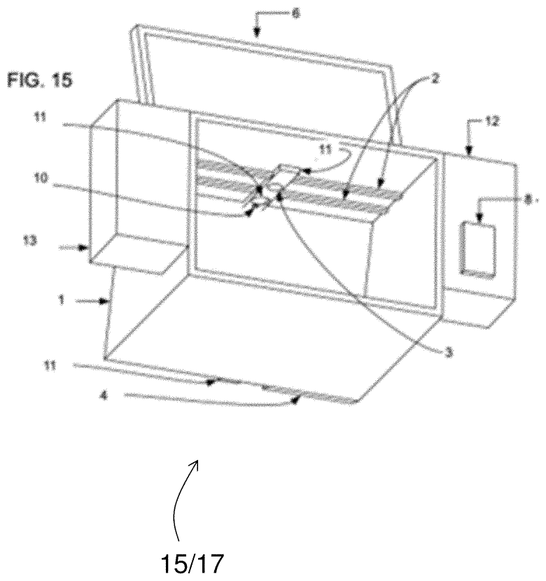

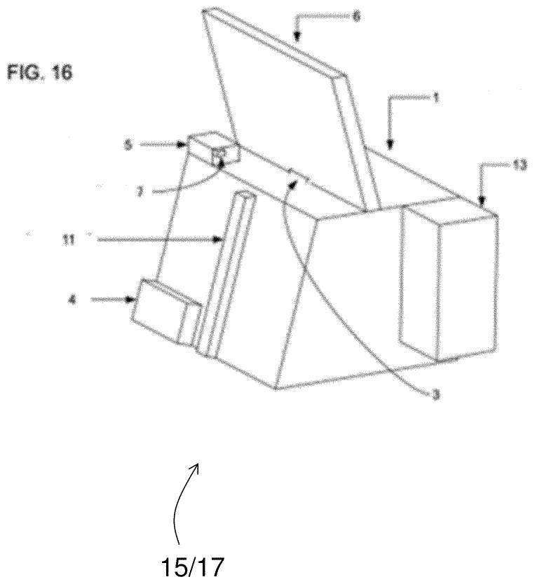

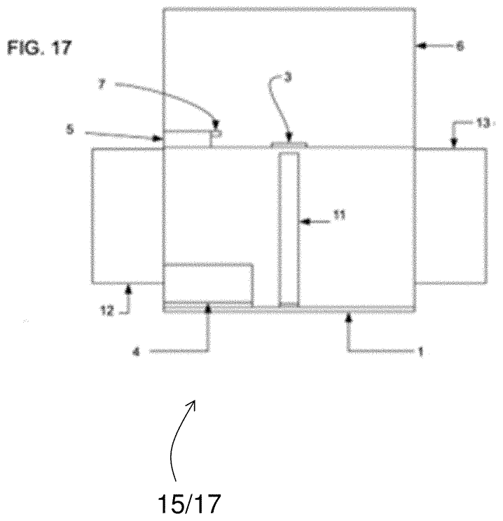

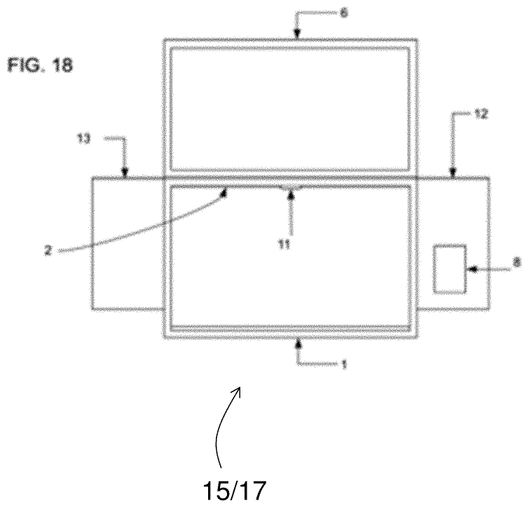

FIG. 16-17 shows a rotated back, left, and top view and back view. In this view, the faucet 11 pipe is shown running down the back wall of the chamber 1. FIG. 18 provides a front view showing the faucet 11, fluorescing agent dispenser 12, and soap dispenser 13.

Overall, the present invention may include various processes that leverage some or all of the components and functionality described to provide various measure of the effectiveness of the sanitization process.

For example, in various embodiments, the system 15 may be provided with power from the power source 4, so the system 15 may be in standby mode and ready for use when activated. A user may activate the system 15 by placing an ID in proximate with the id device 8, by establishing a connection with the wireless device 7, and/or manually by actuating a switch or motion sensor on the system 15.

The system 15 may access, or prompt the user to provide, a user profile that may be stored in the computer 5 and/or a database that may be remote from the computer 5. The system 15 may display information about the user on the display 6, so the user may confirm their identity is correct.

For hand sanitizer application, the next step may involve dispensing the hand sanitizer. The dispenser 9 may be included in the system 15 or may be separate from the system 15. Prior to dispensing the sanitizer, the user may place their hands or the object to be sanitized under the detector 3 so a baseline measurement can be performed. The user then dispenses hand sanitizer onto their hands, rubs in the sanitizer, and places their hands back under the detector 3, so additional measurements can be performed. If the user rubs in the sanitizer under the detector 3, the system 15 may provide real-time updates on the progress of the sanitization.

Sanitization monitoring for hand sanitizers may be provided with the detector 3 being implemented as a camera for detecting a visible indicator and/or a thermal detector for thermal detection, such as changes in the skin temperature due to contact with evaporating hand sanitizers, e.g., alcohol based.

For visual detection using a camera as the detector, the hand sanitizer or soap employed may include, or be used with, a fluorescing substance that fluoresces at the wavelength emitted by the illumination devices 2, e.g., UV-A. For example, the hand sanitizer or soap may include, or be used with, Fluorescein, FD&C Yellow no. 7, which is an FDA approved non-toxic fluorescing dye.

Commercial off the shelf soaps may also be employed in the present invention and used with a fluorescing germ-proxy agent, such as are commercially available under the names Glo-Germ, https://www.glogerm.com/, GlitterBug Potion, https://www.brevis.com/glitterbug, Wash & Glow, and Glow Specialist.

For the embodiments involving soap and water, the user may first dispense the fluorescing germ-proxy agent, if separate, and rub the agent into their hands thoroughly, then dispense soap and rub the soap into their hands thoroughly before rinsing the soap and agent from their hands. In some embodiments, the users may scan their hands, front and back, with the detector 3 after rubbing in the fluorescing germ-proxy agent and before dispensing the soap onto their hands to make sure the fluorescing germ-proxy agent thoroughly covers their hands.

When the user places their hands in the view of the detector 3, which captures images of the user's hands at various stages of the cleaning process, the various images are then compared to assess the effectiveness of the sanitization process. For example, at each stage the user may be instructed to place their hands in certain positions to facilitate the imaging and image comparison process. Videos of the sanitizer application or hand washing, and images of the fluorescing sanitizer's coverage post-application or fluorescing germ-proxy's removal when the user displays their hands flat above the floor of the chamber 1, showing the tops and bottoms, one after the other made be stored and displayed to user. Images may be analyzed by software run in the computer 5, which can both track the user's hand movements to determine how they applied the sanitizers or removed the germ-proxy, and identify hand sanitizing techniques used (which may be those specified by the WHO's or other hand rubbing protocols), as well as determine the absolute level of coverage of the fluorescing sanitizer on the hands or absolute level of removal of the fluorescing germ-proxy.

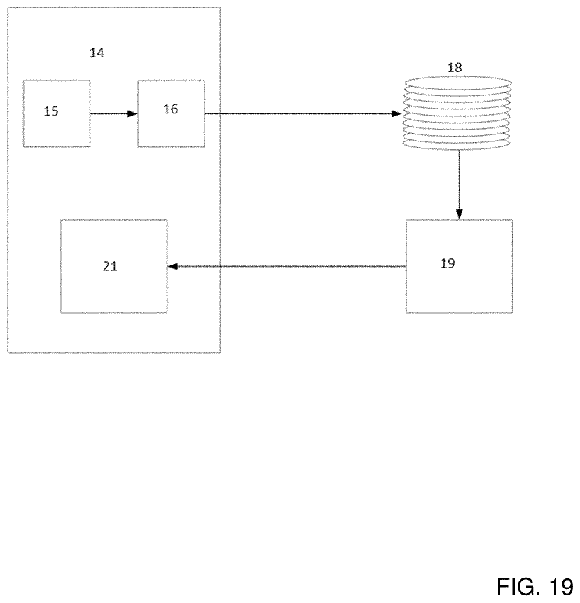

FIG. 19 shows an Ecosystem Map schematic of the system 15 as part of an exemplary ecosystem within which various embodiments may operate. A facility 14 may house the system 15, which in turn, may host some or all of software 16 used by the system 15 to monitor the sanitization process. The software 16 may collect and process information, i.e., raw data and calculated data, from each use of the system 15 and send the information to a database 18, which may be in the cloud, stored locally at the facility 14, and/or hosted elsewhere and may be part of a management system. The information stored in the database 18 may then be accessed by one or more software applications 19 in the cloud, at the facility 14, on the system 15, or elsewhere, which may process the information to make it more understandable, useful, and actionable. This processed data may then be sent to, or accessed by each various facility and/or management platforms 21, whereby users, facility staff (which in the case of healthcare centers may be, but are not limited to, Directors of Nursing, Directors of Epidemiology, Infection Preventionists, Chief Quality Officers, etc.), and other interested parties may access metrics and reports that inform them on the hand sanitization performance and trends of the users, both individually and in the aggregate.

The various components in the system 15 may be controlled by the computer 5, which may be executing some or all of one or more software program on one or more processors and employing various memory/storage devices as described below, to monitor and calculate the effectiveness of the hand cleaning performed by the user. The software implementing the functions, methods, and processes of the present invention may be stored as instructions on transitory and/or non-transitory computer-readable media and executed by one or more processors in the computer 5 as well as remotely, such as in the management system.

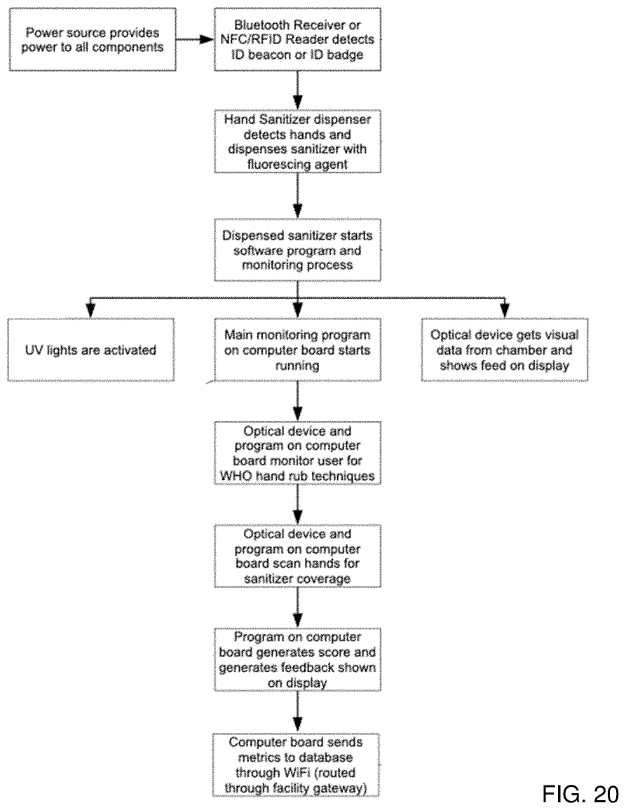

FIG. 20 depicts exemplary component process flows that may be employed in the system 15, such as those depicted in FIGS. 1-18. Initially, power source 4 provides power to all components. Bluetooth 7 or NFC/RFID receiver 8 detects ID beacon or ID badge. Hand Sanitizer dispenser 9 detects hands and dispenses sanitizer with fluorescing agent. Dispensing of the sanitizer may start the software program 16 and monitoring process. Main monitoring program on computer 5 starts running, UV lights 2 are activated, detector 3 collects visual data and provides the data to the computer 5, which process the data and displays it on the display 6. The software may analyze videos or successive images to 1) identify hand rubbing techniques being used, 2) identify the extent of hand coverage during the sanitization process, 3) determine the duration of the sanitization, 4) calculate a score reflective of the coverage, 5) provide the score and optionally feedback to the user via the display, and 6) optionally store information about the sanitization process with the user information on the computer 5 and/or remotely in a database that may be associated with a management system.

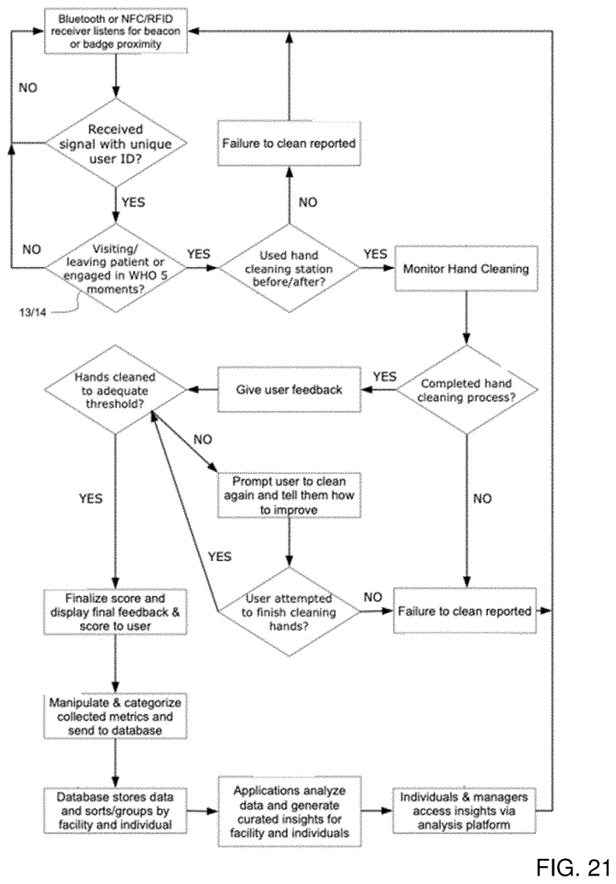

FIG. 21 provides exemplary process flows of the software and component and user interactions, and the experience overall with various embodiments, such as those depicted in FIGS. 1-18. Initially, the Bluetooth or NFC/RFID receiver listens for a beacon or signal from a proximate transmitter. When a signal is received, the software may determine if the signal is from a registered user or not. If not, the software may prompt the user to complete a profile or proceed as a guest. If no response is received to the prompt, the software will return to listening mode and await the next signal. The user may then be identified as someone that needs to engage in hand sanitization, such as people visiting or leaving patients or healthcare workers engaged in the WHO 5 moments of hand hygiene. If not, the software may return to the listening mode. If yes, then the software monitors for the commencement of the hand sanitization process. If the process is not commenced, the software may generate a failure to clean report to be stored including the user information and/or report to a management system and people who monitor cleaning performance. If the process is commenced, the software monitors the sanitization process. If the process is not completed, the software may present an incomplete sanitization message on the display and report and record a failure to complete sanitization.

If the sanitization is completed, the system 15 may provide feedback to the user in terms of tips for improved sanitization and a score reflecting the quality of the sanitization. If the score exceeds a threshold, the user is informed of the successful sanitization and the score and other information from the process recorded, reported, and stored, associated with the user profile. If the score does not exceed a threshold, the user is requested to further sanitize their hands and the system 15 may provide suggestions for improving the score, such as focusing one's cleaning on one's thumbs, fingertips, back of hand, etc, and employing specific techniques to do so. If the user does not complete the sanitization, then the failure is reported and recorded.

The sanitization data may be manipulated & categorized to generate various metrics and stored in the computer and/or sent to a database for remote storage. Reports may be generated and insights provided to various individuals involved with overseeing and improving the sanitization process.

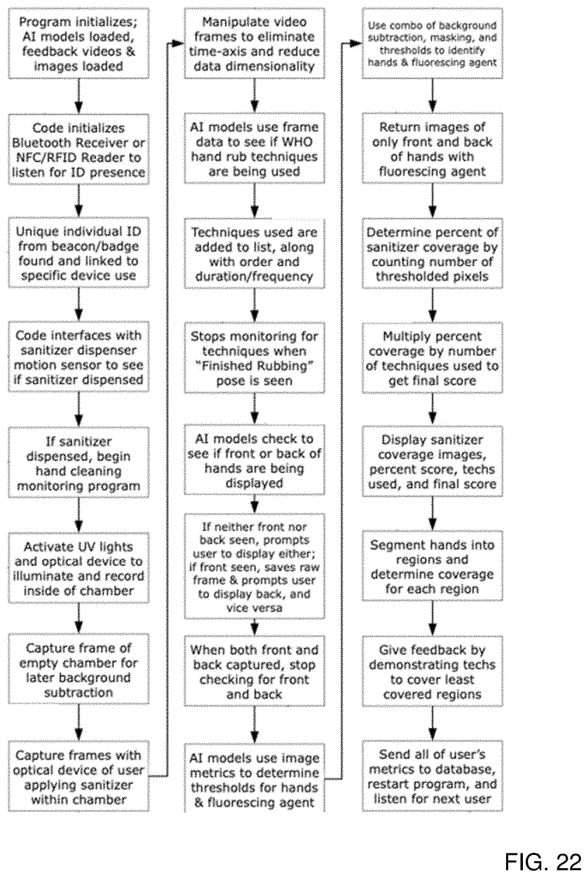

FIG. 22 depicts additional software process flow details that may be employed in the system 15. In step 1, the software initializes; AI models are loaded, feedback videos & images loaded. In step 2, the software initializes Bluetooth receiver and/or NFC/RFID Reader to listen for ID presence. Unique individual ID from beacon/badge or other device may be found and linked to specific device use or new profiles generated for new and guest users. The software may interface with the sanitizer dispenser and any motion sensors to see if sanitizer dispensed. If sanitizer is dispensed, the hand cleaning monitoring program is initialized, activating the UV lights and optical device to illuminate and record activity inside of chamber 1. The software may capture an image (aka frame) of the empty chamber for later background subtraction, if not already or recently stored. Video and/or images of the user applying sanitizer within chamber are then captured.

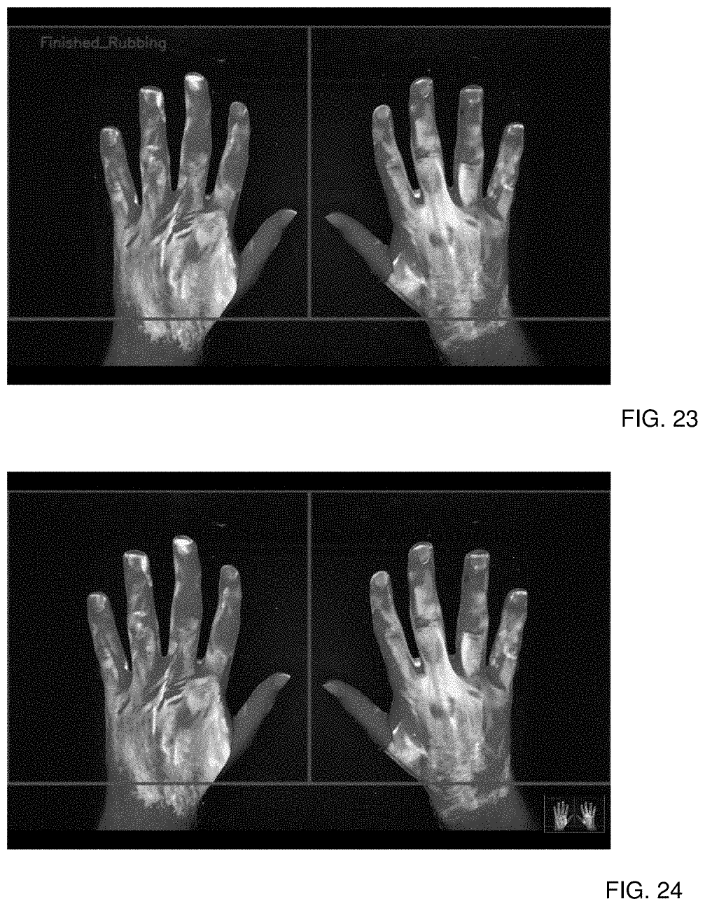

The software may then manipulate video images to eliminate the time-axis and reduce data dimensionality. AI models may use the image data to determine if any World Health Organization (WHO) or other hand rub techniques are being used while the user sanitizes their hands. Techniques used may be added to a list, along with order and duration/frequency of the techniques. The software may stop monitoring when no movement is detected and/or the chamber is empty or when a “finished rubbing” hand position is assumed, e.g., hands separated, top or bottom showing with fingers extended and spread out, see FIG. 23. When the user is finished sanitizing their hands, AI models check to see if either the front or back of hands are being displayed. If neither front nor back of the hand is seen, the software instructions prompt the user to display either side. If the front of the hand is detected, one or more raw images/frames are stored and the user is prompted to display the back of the hand and vice versa. When both front and back images are captured, then software instructions proceed to use AI models or other metrics to compare the images with the thresholds for acceptable sanitization. The software may use background subtraction, masking, and thresholds to identify hands & fluorescing agent. The metrics and AI models may involve various parameters, such as percentage of hand sanitized, locations of sanitized and unsanitized areas, hand washing techniques used, duration, etc. to determine whether the threshold is met and additional instructions and recommendations to provide to the user and management system for immediate and/or future implementation.

The software may display images showing only front and back of hands with fluorescing agent. The software may determine a percent of sanitizer coverage by, for example, counting number of thresholded pixels, and multiply percent coverage by various factors and weighing techniques to get final score.

The hands may be segmented for display and to determine the sanitizer coverage in each hand region for the purposes of giving targeted feedback to the user both in the moment so that they may sanitize said regions to more completely clean their hands, and to the user and system administrators post-cleaning regarding performance trends and potential techniques and strategies to improve sanitization of those regions. Some or all of the data collected during the process may be stored locally in the computer 5 and/or management system that may be local or remote to the system 15 and reported to various personnel.

Other embodiments of the processes and methods described with respect to FIGS. 20-22 may be implemented as well by the skilled artisan. For example, the data collected while the user interacts with the system 15 may be converted into a numerical score, first as an absolute percentage. To do this for the embodiment with the fluorescing agent and visible light camera as the detector 3, the hand is first segmented from the background through background subtraction and thresholding the pixel values of the image, then by thresholding the separate pixel values to determine the amount of sanitizer or fluorescing germ-proxy on one's hand and/or by using the hand tracking to determine the areas rubbed with the sanitizer or soap and the total amount of pixels contained therein. The percentage is then calculated by dividing the amount of pixels thresholded to be sanitizer or fluorescing germ-proxy agent and/or pixels corresponding to areas rubbed by the total amount of pixels thresholded to be the hand. For hand sanitizer, the percentage may represent the amount of coverage, and for hand washing, it may represent the amount of the germ-proxy agent removed.

For the embodiment without a fluorescing agent and a thermal camera as the detector 3, the segmentation of the hand may first show the hands as being warmer than the background, and second show the areas of the hand that are cooler as the areas that are covered by the sanitizer. Determining the percent coverage may be done by counting the number of pixels that fall into the range that represents the cooler sanitizer category. The spots missed by the user may be highlighted and shown on the display 6, alongside the percentage score, etc. The user may be prompted to apply sanitizer or soap again if their score does not meet a high enough threshold value. The software running on the computer 5 may also show techniques on the display 6 that may be used to cover or clean the missed areas, as well as achieve broader coverage or removal overall. The user may be prompted or request, to repeat the process if the score is not as high as desired or if the score does not meet a threshold level. As noted with other embodiments, the raw coverage score may be converted into points, which can be used for gamification and performance tracking to incentivize continued and efficacious use.

With regard to the hand-rubbing techniques, the systems 15 may employ deep learning models to train the software for video gesture recognition that indicate the use of various hand rubbing techniques. The data used to train these models may include videos of both proper and improper execution of various techniques, as well as actions that correspond to no techniques, labeled as such, in order to identify both. Additionally, the video data collected while a player cleans their hands in a use of the device may be collected, labeled, and used to augment the training data, in order to make the models more robust, as well as identify which techniques are most effective, which are easiest to do properly, the best order for best results, etc. While the skilled artisan may implement the video analysis software as desired, it is generally preferable to minimize the computational intensity while still generating highly accurate results, so that low-power, low-cost single board computers 5 may be used running off batteries as the power source 4.

The software will be running in the background while the detector 3 is monitoring the sanitizer application or hand washing, and may log the different techniques used, the amount of repetitions of each technique, and whether or not the techniques were effectively used to achieve proper coverage or removal. When implementing gesture analysis for example, each of the 6 WHO techniques may further be divided into 9 distinct gestures for left & right hand delineation. The scoring multiplier may depend on the number of the gestures used during the sanitization. Instructions on the various techniques may be provided on the display 6 or elsewhere for review by the user.

Once a user has completed the sanitization process, data related to their performance and scoring may be shown on the display (6). As mentioned earlier, the user may be shown the spots they missed on each use of the device, as well as techniques they can use to cover or clean those spots. Additionally, they may be shown what they did correctly as well, to reward and encourage them, with the spots they covered or removed effectively highlighted, and the techniques they used, if any, listed off alongside their multiplier. It may also then show their percentage coverage score and how various multipliers are applied to that score to create their final point score. Finally, some fun graphics may be used to acknowledge their score depending on how the player did, like a smiley emoji, angry emoji, character giving a thumbs up/down or frowning/smiling, etc. The data may be stored locally and/or sent to a remote management system for storage and linked to the user's or guest account. Once the data is transmitted to the database, it can be viewed and analyzed in the analytics platform by the user, as well as the system 15 operators/supervisors at the facility. When the system 15 is not in use, the display 6 may be used to show educational materials, sponsored content, or advertisements.

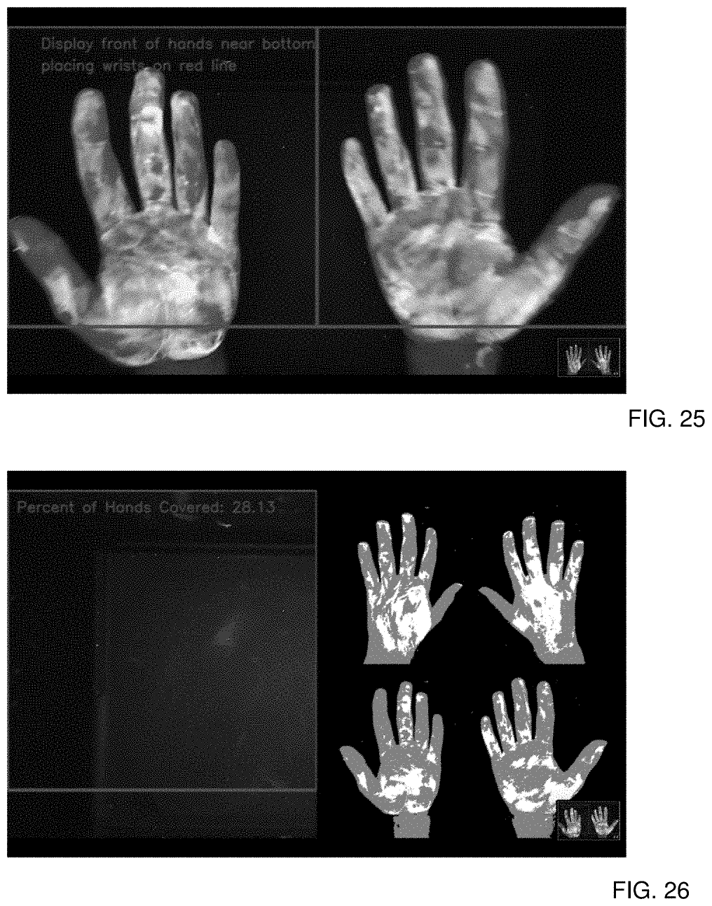

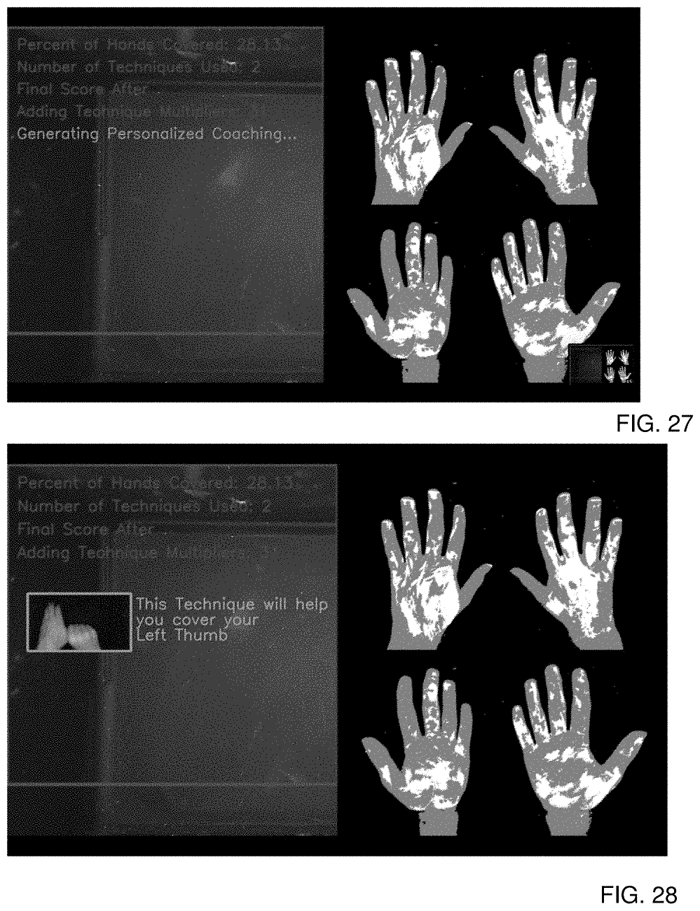

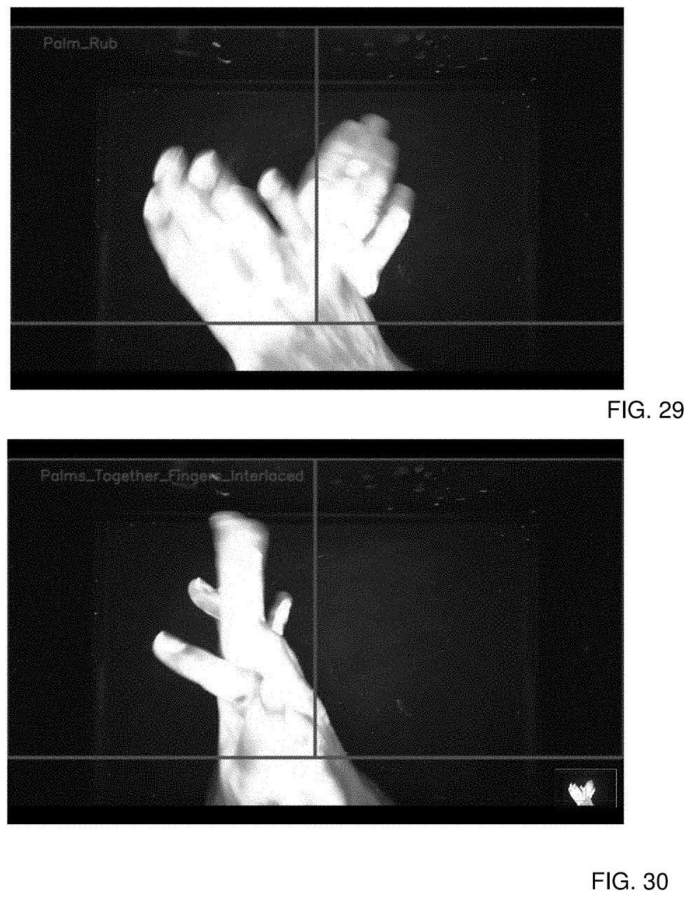

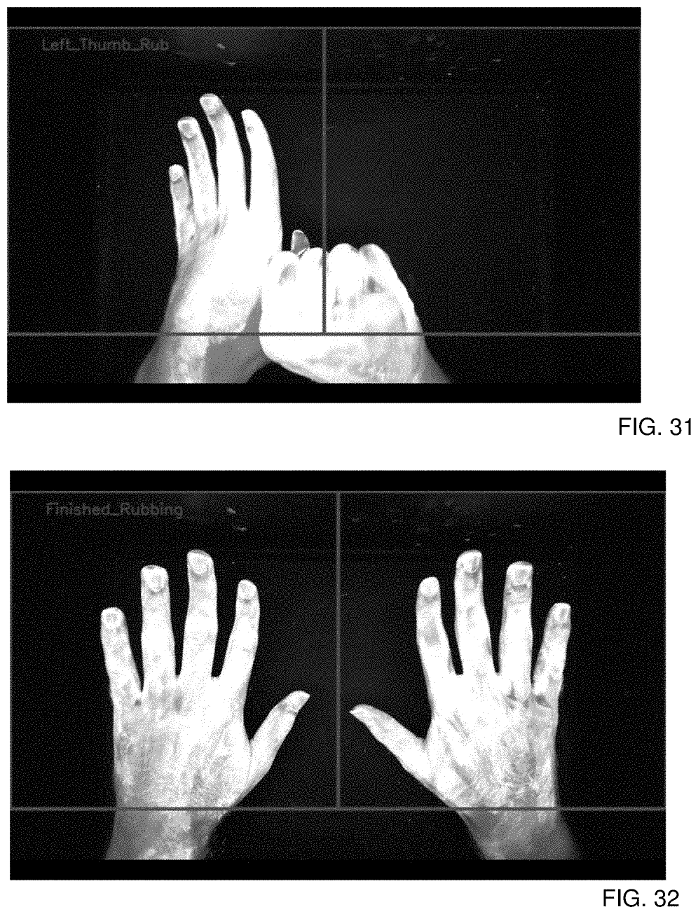

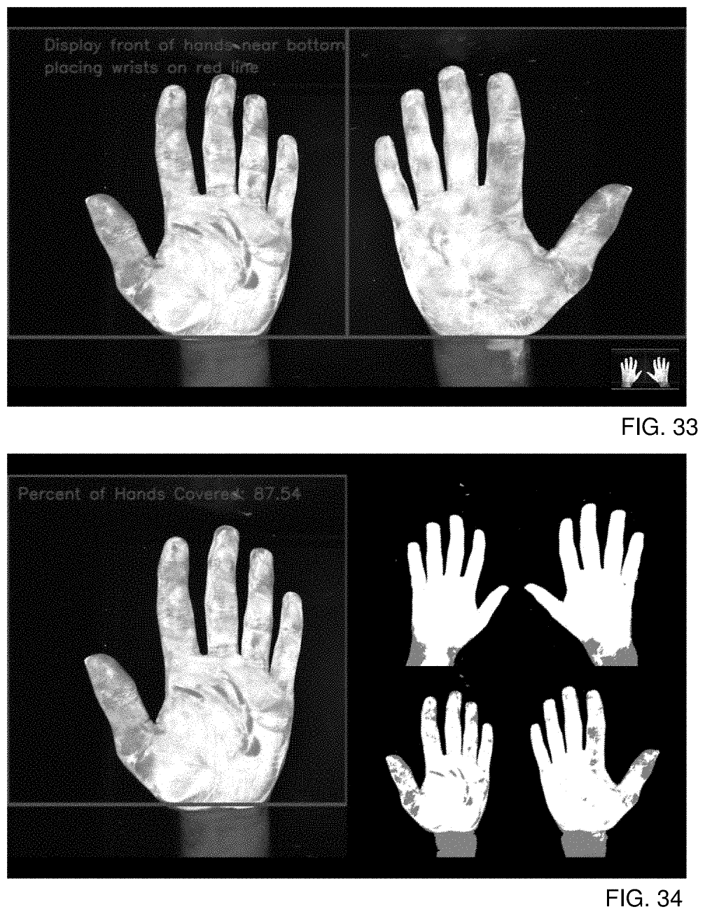

FIGS. 23-34 show exemplary displays involved in the sanitization monitoring and analysis process using the system 15. FIG. 23 shows a user's hands in the finished rubbing or monitoring position. The hands are in the hand detection area with the fingers spread apart to allow the illumination and detection of the hands. FIG. 24 shows the back of the user's hands and the difference in appearance between sanitized and unsanitized areas. FIG. 25 shows the front of the user's hands. The hands are slightly out of the hand detection area, so the system 15 presents the user with a message to move their hands to be better located in the hand detection area, stating “Display front of hands near bottom placing wrists on the red line”. FIG. 26 shows the results of the analysis to the user, namely “Percent of Hands Covered: 28.13” along with images of the user's hands showing sanitized and unsanitized areas. FIG. 27 provides additional information regarding the hand sanitization analysis and informs the user that personalized coaching is being generated based on the analysis. For example, since the user's thumbs were not efficiently sanitized, in FIG. 28, the user is presented with a technique for improving sanitization of the thumbs. FIG. 29 shows a user engaging in a palm rubbing cleaning technique. FIG. 30 shows a user engaging in a palms together fingers interlaced cleaning technique. FIG. 31 shows a user engaging in a thumb cleaning technique. FIG. shows a user with their hands in the finished rubbing or monitoring/inspection position with the back of the hands visible to the detector. FIG. 33 shows a user's hands in the inspection position with the front of the hands visible to the detector.

FIG. 34 shows the results of the analysis, which is an 87.54 percent coverage, which is a dramatic improvement over the initial sanitization attempt. A comparison of FIG. 26 and FIG. 34 is a vivid display of the potential improvement in hygiene that may be brought about by the present invention.

As demonstrated above, systems 15 of the present invention may be used to improve health outcomes by getting individuals to become more invested in their personal hand hygiene, and more motivated and able to ensure they improve their hand hygiene habits and sustain them. Currently, there is no method or technology by which hand cleaning efficacy can be directly measured during the process, thus there is no way for healthcare workers or others to have quantified knowledge and insight into the actual cleanliness of their hands. Most protocols are based on performing the sanitization for a set period of time that may be correlated to hand cleaning efficacy. The present invention may be used to provide real-time insight to improve the process and the level of sanitization.

Hand sanitization may be gamified to further incentivize good sanitization skills and habits by allowing both the user and management to view their results and personal statistics in the analytics platform later. First, a user may link their individual profile to the system 15 and their particular use of the system 15 by identifying themselves at the system 15 as previously described. If the user does not have an existing profile, the user may be prompted to create a profile or proceed as a guest.

As noted, the software running on the computer 5 may monitor the user to determine whether various hand washing techniques are used, e.g., WHO's 6 recommended Hand Rub techniques, and other sanitization measures are performed and the duration. When the sanitization is complete, the software may calculate a percentage coverage sanitization score and then augment the percentage score with additional points for using various techniques, the duration of rubbing, and other measures that may be used to produce a final score (representing both a gamification of the experience, as well as a way to quantify cleaning beyond mere coverage/removal, as the hand rubbing is important as well).

The scoring can depend upon customer preferences, such as awarding no points if their percentage score does not reach a certain coverage threshold, and less points for less than optimal coverage and a lack of techniques, etc. For example, if the percentage score is in the 25th or lower percentile of mean score values, they'll get no points; if they're in the 25th to 75th percentile, they'll get half points; and if they're in the 75th percentile or above, they'll get full points.

Another way to gamify this from the medical facility perspective, which uses percentiles for relative performance incentivization, would be to use the percentiles as qualifications for rewards, instead of not giving users points. For example, users in the top score quartile might get a high monetary bonus, users in the second highest quartile might get a smaller bonus, and users in the bottom two quartiles might not get anything. Or instead of individual compensation, donations to the top users' favorite charities might be made. Furthermore, the percentiles may be dynamic and based on overall users' performance, so that as users continue to improve their hand sanitizing technique and results, achieving the higher percentiles may become progressively harder.

In other embodiments, the scores may be placed along a hyperbolic tangent scale, maybe of the function about 50 tanh(0.05x−2.5)+50 to put it on a 0-100 scale for X (percentage score) and Y (percent of percentage score converted into points), which penalizes scores below the middle value of 50 more so than the values above 50, and where the marginal increase in the percent of points received from the percentage score really levels off as 100 is approached. Other embodiments may be explored to execute this concept of thresholded and/or scaled point awarding. In cases where the user does not achieve full points or close to it, they may be prompted by the display 6 to improve their score by applying sanitizer or soap again, showing areas that were missed, and the techniques that are most helpful in cleaning the user's most missed areas. If they choose to play again and use any recommended techniques, and cover or wash the missed areas, they will be rewarded with a higher score.

Now, like any game, a higher score comes with rewards. In this case, there are a few incentives that will motivate people to get higher scores, and therefore achieve greater hand hygiene. In various embodiments, users may have their average scores and total points earned displayed on leaderboards for the particular facility, area, etc., generated by aggregating the scores of all users, which will give individuals a sense of relative accomplishment. Additionally, individuals may use scores to compete against their friends and colleagues at their facility, inspiring them all to try to clean their hands as well as possible, to rise above their competitors. The public scoring may be displayed as an aggregate for different healthcare worker positions (e.g. nurses vs. doctors vs. surgeons), disciplines (e.g. pediatric vs. geriatrics vs. anesthesiology vs. internal medicine, etc.), shifts, wings of the facility, etc. Another way that points may be used to further incentivise frequent and high-scoring use is to give points value outside of the facility. This may be done by partnering with vendors and stores to make it so points may be redeemed for discounts, coupons, rewards, prizes, etc.

In various embodiments and scenarios, it may be desirable to have penalties for either not using the systems up to the standards set by the facility, or by not getting high enough percent score/point totals. The penalties may be tied into pre-existing disciplinary procedures of the facilities. For example, warnings for individual incidents when a healthcare worker is observed to not engage in hand hygiene activities when they should have, educational materials/classes if a certain amount of warnings have been given, and even a disciplinary writeup to superiors if the individual still hasn't improved their hand hygiene behaviors.

The penalties may focus more on showing the individual their shortcomings and teaching them how to overcome them. For example, generating individual insights that show users when/where/how they don't perform well, what they can do to improve, and reminders to their mobile device/ID device/from their supervisors/etc. to engage in hand hygiene behaviors until they've improved enough to no longer warrant them.

In addition to all the individual profiles and metrics that are part of the experience, the system and the data it collects may be used to create insights on an analytics platform to be used by managers, directors of infection prevention, nursing heads, chief quality/patient safety officers, etc. All scores and other metrics may be linked to the analytics platform, where users, administrators, and others may track performance trends and receive analytics-driven insights. Employers may want to use tracking for compliance, monitoring, and intervention if needed. Examples include, but are not limited to:
Average/instanced hygiene score, as well as running total
Average/instanced % scores
Average/instanced number/type of WHO Hand Rub techniques used
Areas of hand missed most/cleaned best
Compliance & performance before/after visiting patients
Compliance & performance trends throughout HCW shifts
Compliance & performance trends throughout different areas of the facility
Frequency & total number of compliance events
What individual compliance & performance was before interacting with patients that acquired an infection, as well as trends for infection following their interaction
Infection risk scores & recommendations for intervention for individual healthcare workers, wings, shifts, etc, enabled through AI analysis

The analytics platform provides the ability for users to create, view, and take action from insights regarding sanitization practices that are hitherto nonexistent, which may prove invaluable for healthcare facilities in understanding and preventing infections and outbreaks. The present invention takes hand hygiene from an unquantifiable risk to a procedure that can be measured and therefore improved.

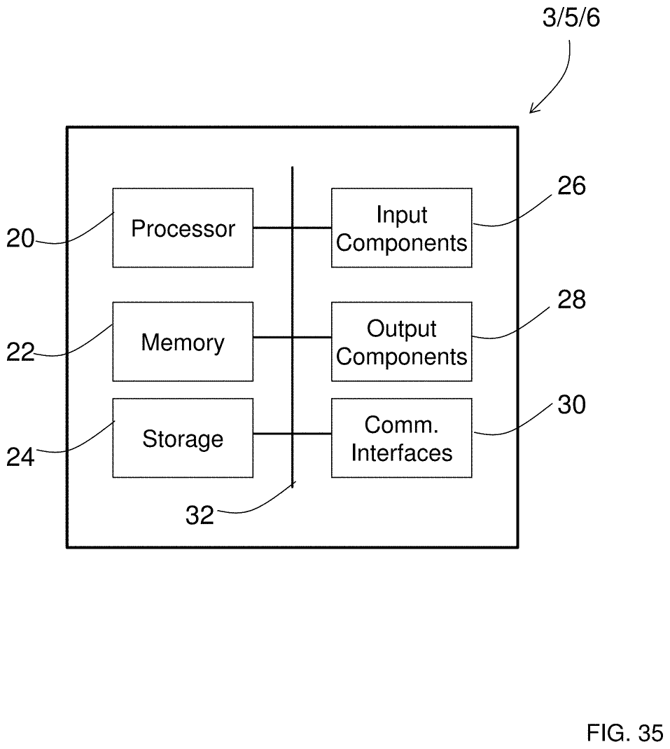

FIG. 35 illustrates exemplary component embodiments of various computing resources, such as computer 5, the detector 3, display 6, illumination device 2, etc., that may be employed in the system 15, and running various applications. The computing resources may each include one or more processors 20, memory 22 and other storage 24, input components 26, output components 28, communication interfaces 30, as well as other components that may be interconnected as desired by the skilled artisan via one or more buses 32. As previously described, the components of the various computing resources may often be configured as a single device or multiple interdependent or stand-alone devices in close proximity and/or distributed over geographically remote areas.

Processor(s) 20 may include one or more general or Central Processing Units (“CPU”), Graphics Processing Units (“GPU”), Accelerated Processing Units (“APU”), microprocessors, and/or any processing components, such as a Field-Programmable Gate Arrays (“FPGA”), Application-Specific Integrated Circuits (“ASIC”), etc. that interpret and/or execute logical functions. The processors 20 may contain cache memory units for temporary local storage of instructions, data, or computer addresses and may be implemented as a single-chip, multiple chips and/or other electrical components including one or more integrated circuits and printed circuit boards that implements and executes logic in hardware, in addition to executing software.

Processor(s) 20 may connect to other computer systems and/or to telecommunications networks as part of performing one or more steps of one or more processes described or illustrated herein, according to particular needs. This can be accomplished through APIs or other methods, using FHIR format or other health-specific format. Moreover, one or more steps of one or more processes described or illustrated herein may execute solely at the processor 20. In addition, or as an alternative, one or more steps of one or more processes described or illustrated herein for execution in one processor may be executed at multiple CPUs that are local or remote from each other across one or more networks.

The computing resources of the system 15 may implement processes employing hardware and/or software to provide functionality via hardwired logic or otherwise embodied in circuits, such as integrated circuits, which may operate in place of or together with software to execute one or more processes or one or more steps of one or more processes described or illustrated herein. Software implementing particular embodiments may be written in any suitable programming language (e.g., procedural, object oriented, etc.) or combination of programming languages, where appropriate.

Storage may include various types of memory 22, e.g., Random Access Memory (“RAM”), Read Only Memory (“ROM”), and/or another type of dynamic or static memory devices, such as flash, magnetic, and optical memory, etc. that stores information and/or instructions for use by processor 20. The memory 22 may include one or more memory cards that may be loaded on a temporary or permanent basis. Memory 22 and storage 24 may include a Subscriber Identification Module (“SIM”) card and reader.

Other storage components 24 may be used to store information, instructions, and/or software related to the operation of the system 15 and computing resources. Storage 24 may be used to store operating system, executables, data, applications, and the like, and may include fast access primary storage, as well as slower access secondary storage, which may be virtual or fixed.

Storage component(s) 24 may include one or more transitory and/or non-transitory computer-readable media that store or otherwise embody software implementing particular embodiments. The computer-readable medium may be any tangible medium capable of carrying, communicating, containing, holding, maintaining, propagating, retaining, storing, transmitting, transporting, or otherwise embodying software, where appropriate, including nano-scale medium. The computer-readable medium may be a biological, chemical, electronic, electromagnetic, infrared, magnetic, optical, quantum, or other suitable medium or a combination of two or more such media, where appropriate. Example computer-readable media include, but are not limited to fixed and removable drives, ASIC, Compact Disks (“CDs”), Digital Video Disks (“DVDs”), FPGAs, floppy disks, optical and magneto-optic disks, hard disks, holographic storage devices, magnetic tape, caches, Programmable Logic Devices (“PLDs”), Secure Disk Cards (“SD Cards”), RAM devices, ROM devices, semiconductor memory devices, solid state drives, cartridges, and other suitable computer-readable media.

Input components 26 and output components 28 may include various types of Input/ Output (“I/O”) devices. The I/O devices often may include a Graphical User Interface (“GUI”) that provides an easy to use visual interface between the user and system 15 and access to the operating system or application(s) running on the devices.

Input components 26 receive any type of input in various forms from users or other machines, such as touch screen and video displays, keyboards, keypads, mice, buttons, track balls, switches, joy sticks, directional pads, microphones, cameras, transducers, card readers, voice and handwriting inputs, and sensors for sensing information such as biometrics, temperature & other environmental conditions, such as air quality, etc., location via Global Positioning System (“GPS”) or otherwise, accelerometer, gyroscope, compass, actuator data, which may be input via a component in the computing resource and/or received via one or more communication interfaces 30.

Output component 28 may include displays, speakers, lights, sensor information, mechanical, or other electromagnetic output. Similar to the input, the output may be provided via one or more ports and/or one or more communication interfaces 30.

Communication interface 30 may include one or more transceivers, receivers, transmitters, modulators, demodulators that enable communication with other devices, via wired and/or wireless connections. Communication interface 30 may include Ethernet, optical, coaxial, Universal Serial Bus (“USB”), Infrared (“IR”), Radio Frequency (“RF”) including the various Wi-Fi, WiMax, cellular, and Bluetooth protocols, such as Bluetooth, Bluetooth Low Energy (BLE), Wi-Fi (IEEE 802.11), Wi-Fi Direct, SuperWiFi, 802.15.4, WiMax, LTE systems, LTE Direct, past, current, and future cellular standard protocols, e.g., 4-5G, or other wireless signal protocols or technologies as described herein and known in the art.

Bus(es) 32 may connect a wide variety of other subsystems, in addition to those depicted, and may include various other components that permit communication among the components in the computing resources. The bus(es) 32 may encompass one or more digital signal lines serving a common function, where appropriate, and various structures including memory, peripheral, or local buses using a variety of bus architectures. As an example and not by way of limitation, such architectures include an Industry Standard Architecture (“ISA”) bus, an Enhanced ISA (“EISA”) bus, a Micro Channel Architecture (“MCA”) bus, a Video Electronics Standards Association Local Bus (“VLB”), a Peripheral Component Interconnect (“PCI”) bus, a PCI-eXtended (“PCI-X”) bus, a Peripheral Component Interconnect Express (PCIe) bus, a Controller Area Network (“CAN”) bus, and an Accelerated Graphics Port (“AGP”) bus.

The computing resources of the system 15 may provide functionality as a result of the processors 20 executing software embodied in one or more computer-readable storage media residing in the memory 22 and/or storage 24 and logic implemented and executed in hardware. The results of executing the software and logic may be stored in the memory 22 and/or storage 24, provided to output components 28, and transmitted to other devices via communication interfaces 30, which includes cloud storage and cloud computing. In execution, the processor 20 may use various inputs received from the input components 26 and/or the communications interfaces 30. The input may be provided directly to the processor 20 via the bus 32 and/or stored before being provided to the processor 20. Executing software may involve carrying out processes or steps may include defining data structures stored in memory 22 and modifying the data structures as directed by the software.

As used herein, the term component is intended to be broadly construed as hardware, firmware, and/or a combination of hardware and software. It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware can be designed to implement the systems and/or methods based on the description herein.

Certain user interfaces have been described herein and/or shown in the figures. A user interface may include a graphical user interface, a non-graphical user interface, a text-based user interface, etc. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

Some implementations are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

The foregoing disclosure provides examples, illustrations and descriptions of the present invention, but is not intended to be exhaustive or to limit the implementations to the precise form disclosed. Modifications and variations are possible in light of the above disclosure or may be acquired from practice of the implementations. These and other variations and modifications of the present invention are possible and contemplated, and it is intended that the foregoing specification and the following claims cover such modifications and variations.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items and may be used interchangeably with “one or more.” Furthermore, as used herein, the term “set” is intended to include one or more items and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A hand sanitization monitoring system comprising:
a chamber;
at least one illumination device positioned within the chamber to illuminate a hand detection area within the chamber;
a detector positioned within the chamber to detect and provide images of hands within the hand detection area;
at least one processor and a memory positioned proximate the chamber, the processor to
receive the images from the detector,
determine areas of the image corresponding to sanitized areas of the hands from unsanitized areas of the hands,
calculate a score using at least a percentage of the sanitized areas to a total area corresponding to the sanitized area plus the unsanitized area, and
provide at least the score; and
a display positioned proximate the chamber to display at least the score provided by the processor.

2. The system of claim 1, where
the at least one illumination device is at least one UV light; and
the detector is a camera capturing visual images of the hands.

3. The system of claim 1, further comprising
at least one of a hand sanitizer dispenser containing fluorescing hand sanitizer and a fluorescing germ-proxy agent with soap dispenser positioned proximate the chamber, where
the processor calculates the sanitized area based on the fluorescence of the hands and provides instructions on using the system to sanitize their hands with the fluorescing hand sanitizer and a fluorescing germ-proxy agent with soap to the display for viewing.

4. The system of claim 1, where
the detector is a thermal imaging camera capturing thermal images of the hands.

5. The system of claim 1, where
the processor is further to
determine at least one of hand rubbing techniques and a duration of hand rubbing from the images; and
calculate the score using at least one of the detected hand rubbing techniques and the duration of hand rubbing.

6. The system of claim 5, where
the processor is further to
identify a user of the system based on information from at least one of an identification device, information input into the display, and an identifier received via a wireless signal;
store at least one of the score, the percentage of sanitized area, the detected hand rubbing techniques, and the duration of hand rubbing with the user identity; and
provide at least one of the score, the percentage of sanitized area, the detected hand rubbing techniques, and the duration of hand rubbing to the display for viewing.

7. The system of claim 6, where
the processor is further to
compare information including at least one of the score, percentage of sanitized area, detected hand rubbing techniques, and duration of hand rubbing of the user with the information of groups of users including at least one other user; and
provide the comparison of information to the display for viewing.

8. The system of claim 6, where
the processor is further to
request, via the display, the user to
further sanitize their hands when the score does not exceed a threshold score; and
place their hands in the hand detection area to be detected at least following further sanitization.

9. The system of claim 1, where
the processor is further to
associate at least the score with a user; and
store the score associated with the user.

10. The system of claim 9, where
the processor is further to
transmit the score and associated user to a remote management system.

11. The system of claim 1, where
the processor is further to
receive background images of the chamber without hands in the hand detection area; and;
subtract the background images from the images of the hands.

12. The system of claim 1, further comprising:
a water faucet to dispense water proximate the hand detection area.

13. A method hand sanitization monitoring comprising:
providing a chamber with at least one illumination device positioned within the chamber to illuminate a hand detection area within the chamber, and a detector positioned within the chamber to detect and provide images of hands within the hand detection area;
instructing a user to dispense at least one of a fluorescing hand sanitizer and a fluorescing germ-proxy agent with soap onto their hands and place their hands in the hand detection area;
illuminating, by the at least one illumination device; their hands placed in the hand detection area;
detecting and providing images of the hands by the detector;
receiving, by a processor, the images from the detector;
determining, by the processor, areas of the image corresponding to sanitized areas of the hands from unsanitized areas of the hands based on the fluorescence of the hands,
calculating, by the processor, a score using at a percentage of sanitized areas to the total area corresponding to the sanitized and unsanitized areas, and
providing, by the processor, at least the score to at least a display; and
displaying, by the display, at least the score.

14. The method of claim 13, further comprising:
instructing the user to rub the at least one of a fluorescing hand sanitizer and a fluorescing germ-proxy agent with soap onto their hands in the hand detection area, and the method further comprises:
detecting at least one of hand rubbing techniques and a duration of hand rubbing from the images; and
calculating the score using at least one of the detected hand rubbing techniques and the duration of hand rubbing.

15. The method of claim 14, further comprising:
identifying a user based on information from at least one an identification device, information input into the display, and an identifier received via a wireless signal;
storing at least one of the score, the percentage of sanitized area, the detected hand rubbing techniques, and the duration of hand rubbing; and
providing at least one of the score, the percentage of sanitized area, the detected hand rubbing techniques, and the duration of hand rubbing to the display for viewing.

16. The method of claim 15, where
comparing information include at least one of the score, percentage of sanitized area, detected hand rubbing techniques, and duration of hand rubbing of the user with the information of groups of user including at least one other user; and
providing the comparison of information to the display for viewing.

17. The method of claim 13, where
requesting, via the display, the user to
further sanitize their hands when the score does not exceed a threshold score; and
place their hands in the hand detection area to be detected following further sanitization.

18. The method of claim 13, where
calculating the score is performed by counting pixels in the image corresponding to sanitized areas and unsanitized areas.

19. A sanitization monitoring system comprising:
a detector to provide at least one image of an object within its detection range; and
at least one processor, the processor to
receive the at least one image from the detector,
determine areas of the image corresponding to sanitized areas of the object from unsanitized areas of the object,
calculate a percentage of sanitized areas to the total area corresponding to the sanitized and unsanitized areas, and
report at least the percentage of sanitized area.

20. The system of claim 19, where
the object is at least one human hand; and
the detector is a thermal imaging camera positioned to detect temperature differences on the at least one human hand resulting from hand sanitizer contacting the hands.
